(19) United States
(12) Patent Application Publication
(10) Pub. No.: US 2005/0124579 A1
(43) Pub. Date: Jun. 9, 2005

(54) Benzyl alcohol derivatives

(76) Inventors: Werner Holzl, Eschentzwiller (FR); Jurgen Koppold, Lorrach (DE); Sophie Marquais-Bienewald, Hegenheim (FR); Andrea Preuss, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/507,965

(22) PCT Filed: Mar. 13, 2003
(86) PCT No.: PCT/EP03/02618

(30) Foreign Application Priority Data
Mar. 19, 2002 (EP) 02405210.2

(51) Int. Cl. A61K 31/695; C07F 7/04; A61K 31/075

(52) U.S. Cl. 514/63; 514/718; 514/734; 556/449; 568/662

(57) ABSTRACT

The use of benzyl alcohol derivatives of the formula CH2OH in which R, R2, R3, n and m have the meanings given in claim 1, as microbicidal active substances is described. The compounds exhibit a marked effect against pathogenic Gram-positive and Gram-negative bacteria. They are therefore suitable for the antimicrobial treatment, in particular preservation and disinfection, of surfaces.

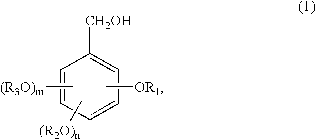

The present invention relates to the use or benzyl alcohol derivatives for the antimicrobial treatment of surfaces, as antimicrobial active substance against Gram-positive and Gram-negative bacteria, and preservation of cosmetics, household products, textiles, plastics and disinfectants, and also to novel benzyl alcohol derivatives.

The benzyl alcohol derivatives used according to the invention have the formula 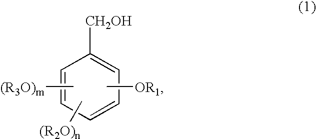
in which
R₁,R₂ and R₃, independently of one another, are unsubstituted C₁-C₂₀alkyl, C₅₋₈cycloalkyl, C₃-C₂₀alkenyl or C₁-C₂₀alkyl, C₅₋₈cycloalkyl, C₃-C₂₀alkenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; C₂-C₂₀alkyl which is interrupted by one or more heteroatoms and/or may be substituted; C₂-C₂₀perfluoroalkyl; —(C₂-C₁₂alkylene)-Si-(tris-C₁₋₁₂alkyl); —(C₂-C₁₂alkylene)-Si-(di-C₁₋₁₂alkyl)-C₃-C₁₂alkenyl; or —(CH₂)ₓ(CHCH₃)_{y}(C(CH₃)₂)_{z}-A;
A is unsubstituted phenyl, naphthyl or C₄-C₈cycloalkyl or phenyl, naphthyl or C₄-C₈cycloalkyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; or —CH₂CH₂(CH₂CH₂O)ₒ—B;
B is unsubstituted C₁-C₄alkyl or phenyl or C₁-C₄alkyl or phenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂;
o is an integer from 0-10;
n and m, independently of one another, are 0 or 1; and
x, y and z, independently of one another, are 0 to 12;
and mixtures thereof and salts thereof,
where compounds of the formula (1) in which
R₁ and R₂ or R₁ and R₃ or R₂ and R₃ are methyl or ethyl are not included.

The C₂-C₂₀alkyls interrupted by one or more heteroatoms are interrupted, in particular, by O, S or N.
C₁-C₂₀Alkyls are straight-chain or branched alkyl radicals, such as, for example, methyl, ethyl, n-propyl, isopropyl, n-butyl, sec-butyl, tert-butyl, pentyl, hexyl, heptyl, octyl, nonyl, decyl, undecyl, dodecyl, tetradecyl, pentadecyl or hexadecyl.
C₁-C₄Alkoxy is, for example, methoxy, ethoxy, n-propoxy, isopropoxy or n-butoxy. Halogen is fluorine, chlorine, bromine or iodine.

In preferred compounds used according to the invention,
R₁, R₂ and R₃, independently of one another, are unsubstituted C₅-C₁₆alkyl, C₃-C₆cycloalkyl or C₃-C₁₆alkenyl or C₅-C₁₆alkyl, C₃-C₆cycloalkyl or C₃-C₁₆alkenyl substituted by C₁-C₄alkyl, C₁-C₄alkoxy, hydroxy, phenyl, halogen, CN, COOH, COO—C₁-C₂-alkyl, amino, NHC₁-C₂₀alkyl, N(C₁-C₂₀alkyl)₂; C₃-C₁₆alkyl which is interrupted by one or more heteroatoms from the group O, N or S; C₁-C₁₆perfluoroalkyl; —(C₁-C₁₂alkylene)-Si-(tris-C₁₋₁₂alkyl); —(C₁-C₆alkylene)-Si-(di-C₁-C₂alkyl)allyl; —(CH₂)ₓ(CHCH₃)_{y}(C(CH₃)₂)_{z}-A; or —CH₂CH₂(CH₂CH₂O)ₒ—B;
A is unsubstituted phenyl or C₅-C₆cycloalkyl or phenyl or C₅-C₆cycloalkyl substituted by C₁-C₄alkyl, C₁-C₄alkoxy, hydroxy, phenyl, halogen, —CN, —COOH, —COO—C₁-C₂alkyl, amino, NHC₁-C₂₀alkyl or N(C₁-C₂₀alkyl)₂;
B is unsubstituted C₁-C₂alkyl or phenyl or C₁-C₂alkyl or phenyl substituted by C₁-C₄alkyl, C₁-C₄alkoxy, hydroxy, phenyl, halogen, —CN, —COOH, —COO—C₁-C₂alkyl, amino, NHC₁-C₂₀alkyl or N(C₁-C₂₀alkyl)₂;
x, y and z, independently of one another, are an integer from 0 to 4;
o is an integer from 0 to 5; and
n and m, independently of one another, are 0 or 1.

Interesting compounds which can be used according to the invention have the formula 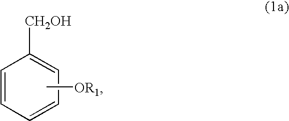
in which
R₁ is —(CH₂)₁₋₃Si(CH₃)₃; —CH₂Si(CH₃)₂(CH₂CH═CH₂); —CH₂SiC₆H₅; —(CH₂)₅CH₃; —(CH₂)₇CH₃; —CH(CH₂CH₃)₂; or —CH(CH₃)(CH₂)₄CH₃.

Further interesting compounds which can be used according to the invention have the formulae 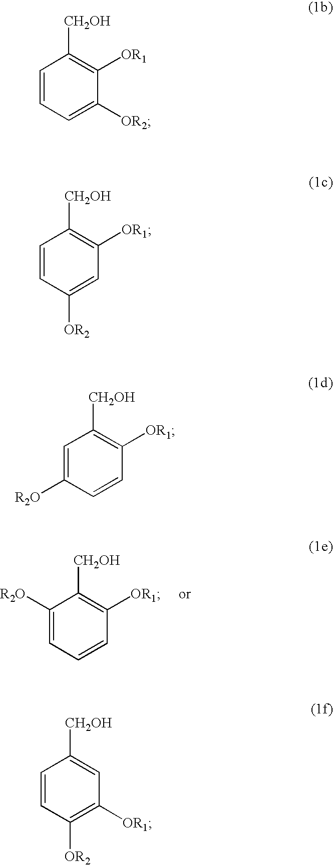
in which
R₁ is —(CH₂)₁₋₃Si(CH₃)₃; —CH₂Si(CH₃)₂(CH₂CH═CH₂); —CH₂Si(CH₃)₂C₆H₅; linear C₄-C₁₀alkyl; —CH(CH₂CH₃)₂; —CH(CH₃)(CH₂)₄CH₃; —CH₂CH₂OC₆H₅; cyclohexyl; or —CH₂C₆H₅;
R₂ is —C₁-C₄alkyl; —(CH₂)₁₋₃Si(CH₃)₃; —CH₂Si(CH₃)₂(CH₂CH═CH₂); —CH₂Si(CH₃)₂C₆H₅; —(CH₂)₃CH₃; —(CH₂)₅CH₃; —(CH₂)₇CH₃; —CH(CH₂CH₃)₂; —CH(CH₃)(CH₂)₄CH₃; —CH₂CH₂OC₆H₅; cyclohexyl; or —CH₂C₆H₅.

Particularly preferred compounds of the formulae (1b)-(1f) are those in which
R₁ is —CH₂Si(CH₃)₃—(CH₂)₃Si(CH₃)₃; —CH₂Si(CH₃)₂(CH₂CH═CH₂); —CH₂Si(CH₃)₂C₆H₅; —(CH₂)₅CH₃; —(CH₂)₇CH₃; —CH(CH₂CH₃)₂; —CH(CH₃)(CH₂)₄CH₃; —CH₂CH₂OC₆H₅; —(CH₂)₃CH₃; cyclohexyl; or —CH₂C₆H₅; and
R₂ is methyl.

Table 1 below lists examples of further compounds which can be used according to the invention:

TABLE 1

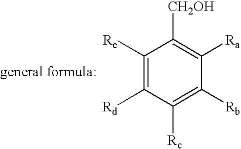

| Comp. | | | | | |
|---|---|---|---|---|---|
| of the | | | | | |
| for- | | | | | |
| mula | Rₐ | R_{b} | R_{c} | R_{d} | Rₑ |
| (3) | —O(CH₂)₃Si(CH₃)₃ | H | H | H | H |
| (4) | —OCH₂Si(CH₃)₃ | H | H | H | H |
| (5) | —O(CH₂)₇CH₃ | H | H | H | H |
| (6) | —OCH(CH₂CH₃)₂ | H | H | H | H |
| (7) | —OCH(CH₃)(CH₂)₄CH₃ | H | H | H | H |
| (8) | H | H | —O(CH₂)₃Si(CH₃)₃ | H | H |
| (9) | H | H | —O(CH₂)₂O(CH₂)₂OCH₃ | H | H |
| (10) | H | H | —O(CH₂)₅CH₃ | H | H |
| (11) | H | H | —O(CH₂)₇CH₃ | H | H |
| (12) | H | —O(CH₂)₃Si(CH₃)₃ | H | H | H |
| (13) | H | —OCH₂Si(CH₃)₂C₆H₅ | H | H | H |
| (14) | H | —O(CH₂)₇CH₃ | H | H | H |
| (15) | H | —OCHCH₃(CH₂)₄CH₃ | H | H | H |
| (16) | H | —OCH₃ | H | H | —O(CH₂)₃Si(CH₃)₃ |
| (17) | H | —OCH₃ | H | H | —OCH₂Si(CH₃)₃ |
| (18) | H | —OCH₃ | H | H | —OCH₂CH₂OC₆H₅ |
| (19) | H | —OCH₃ | H | H | —O(CH₂)₃CH₃ |
| (20) | H | —OCH₃ | H | H | —O(CH₂)₅CH₃ |
| (21) | H | —OCH₃ | H | H | —O(CH₂)₇CH₃ |
| (22) | H | —OCH₃ | H | H | —OCH(CH₂CH₃)₂ |
| (23) | H | —OCH₃ | H | H | —OCHCH₃(CH₂)₄CH₃ |
| (24) | H | —OCH₃ | H | H | 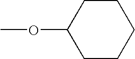 |
| (25) | H | —OCH₃ | H | H | —OCH₂C₆H₅ |
| (26) | —OCH₃ | H | H | H | —O(CH₂)₇CH₃ |
| (27) | —O(CH₂)₇CH₃ | —OCH₃ | H | H | H |
| (28) | H | H | —O(CH₂)₇CH₃ | —OCH₃ | H |
| (29) | H | —OCH₂Si(CH₃)₂CH₂CH═CH₂ | —OCH₂Si(CH₃)₂CH₂CH═CH₂ | H | H |
| (30) | H | —O(CH₂)₇CH₃ | —O(CH₂)₇CH₃ | H | H |
| (31) | H | —O(CH₂)₁₁CH₃ | —O(CH₂)₁₁CH₃ | H | H |
| (32) | —O(CH₂)₁₁CH₃ | H | —O(CH₂)₁₁CH₃ | H | H |
| (33) | 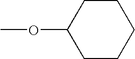 | H | 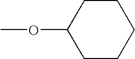 | H | H |
| (34) | —O(CH₂)₇CH₃ | H | H | —O(CH₂)₇CH₃ | H |
| (35) | —O(CH₂)₁₁CH₃ | H | H | —O(CH₂)₁₁CH₃ | H |
| (36) | H | —O(CH₂)₇CH₃ | H | H | H |
| (37) | H | H | —O(CH₂)₇CH₃ | H | H |
| (38) | —O(CH₂)₇CH₃ | H | H | —OCH₃ | H |
| (39) | —O(CH₂)₇CH₃ | H | H | H | —OCH₃ |
| (40) | H | —O—CH₃ | —O(CH₂)₁₁CH₃ | —O—CH₃ | H |

Preferred are the compounds of formulae (14), (28), (37), (38), (39) and (40).

Benzyl alcohol derivatives used according to the invention are prepared by known methods, which comprises reacting a compound of the formula (2a) 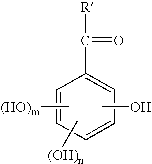
with a halide Hal-R₁ or a mixture of Hal-R₁/Hal-R₂, Hal-R₁/Hal-R₃, Hal-R₂/Hal-R₃ or Hal-R₁/Hal-R₂/Hal-R₃, in which all substituents can have the meanings given in formula (1) and R′ is hydrogen or O—C₁-C₅alkyl, to give a compound of the formula (2b)

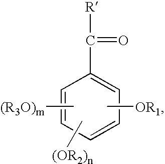

The compound of the formula (2b) is then converted, by reduction, into the compound of the formula (1).

The overall reaction can be represented by the following scheme:

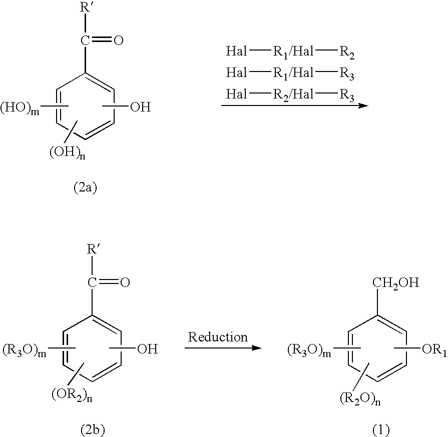

The synthesis of the compounds of the formula (2b) is, in particular, carried out in a solvent, such as dimethylformamide, methanol, ethanol, acetone, acetonitrile or ethyl acetate, to which an (auxiliary) base, such as, for example, sodium hydride, sodium alkoxides or alkali metal carbonate, has been added, at a temperature of from 40 to 120° C., preferably 60 to 100° C.

Per OH group of the compound of the formula (2a), preferably 1.1 equivalent of the halide Hal-R₁ or a mixture of Hal-R₁/Hal-R₂, Hal-R₁/Hal-R₃, Hal-R₂/Hal-R₃ or Hal-R₁/Hal-R₂/Hal-R₃ is added. Preferred halides are chlorides and bromides.

The reaction time is 2 to 48 hours. A preferred reaction time is about 18 hours.

The reduction of the compounds of the formula (2b) to give compounds of the formula (1) is carried out in accordance with customary methods.

The reducing agents used are customary reducing agents, such as, for example, hydrogen, metal hydrides or dithionite.

The solvents used are, for example, diethyl ether or tetrahydrofuran.

The reducing agent is customarily added in a 10- to 20-fold excess.

The reaction temperature is usually between 20 and 50° C., preferably 25 to 45° C.

Further details relating to the preparation process are given in the corresponding examples.

The benzyl alcohols used according to the invention exhibit a marked antimicrobial effect, in particular against pathogenic Gram-positive and Gram-negative bacteria and also against skin flora bacteria. They are therefore suitable, in particular, for the disinfection, deodorization, and also the general and antimicrobial treatment of the skin and mucosae, and skin appendages (hair), very particularly for hand and wound disinfection. They are therefore suitable as antimicrobial active substances and preservatives in bodycare compositions, such as, for example, shampoos, bath products, haircare compositions, liquid and solid soaps (based on synthetic surfactants and salts of saturated and/or unsaturated fatty acids), lotions and creams, deodorants, other aqueous or alcoholic solutions, e.g. cleansing solutions for the skin, moist cleansing wipes, oils or powders.

The invention therefore further provides a bodycare composition comprising at least one compound of the formula (1) and cosmetically acceptable carriers or auxiliaries.

The bodycare composition according to the invention comprises 0.01 to 15% by weight, preferably 0.1 to 10% by weight, based on the total weight of the composition, of the benzyl alcohol compound of the formula (I) and cosmetically acceptable auxiliaries.

Depending on the form in which the bodycare composition is present, as well as the benzyl alcohol compound of the formula (1), it also has further constituents, such as, for example, sequestering agents, dyes, perfume oils, thickening or setting agents (consistency regulators), emollients, UV-absorbers, skin protectants, antioxidants, additives which improve the mechanical properties, such as dicarboxylic acids and/or Al, Zn, Ca, Mg salts of C₁₄-C₂₂ fatty acids and optionally preservatives.

The bodycare composition according to the invention can be formulated as a water-in-oil emulsion or oil-in-water emulsion, as an alcoholic or alcohol-containing formulation, as a vesicular dispersion of an ionic or nonionic amphiphilic lipid, as a gel, solid stick or as an aerosol formulation.

As a water-in-oil or oil-in-water emulsion, the cosmetically acceptable auxiliary preferably comprises 5 to 50% of an oil phase, 5 to 20% of an emulsifier and 30 to 90% of water. The oil phase can comprise any oil suitable for cosmetic formulations, such as, for example, one or more hydrocarbon oils, a wax, a natural oil, a silicone oil, a fatty acid ester or a fatty alcohol. Preferred mono- or polyols are ethanol, isopropanol, propylene glycol, hexylene glycol, glycerol and sorbitol.

Cosmetic formulations according to the invention are used in various fields. In particular, the following compositions, for example, are considered:
skincare compositions, such as, for example, skin washing and cleansing compositions in the form of bar or liquid soaps, syndets or washing pastes,
bath preparations, such as, for example, liquid bath preparations (foam baths, milks, shower preparations) or solid bath preparations, such as, for example, bath tablets and bath salts;
skincare compositions, such as, for example, skin emulsions, multiple emulsions or skin oils;
decorative bodycare compositions, such as, for example, make-up for the face in the form of day or powder creams, face powder (loose and pressed), blusher or cream
make-up, eyecare compositions, such as, for example, eyeshadow preparations, mascara, eyeliner, eye creams or eye-fix creams; lipcare compositions, such as, for example, lipstick, lip gloss, lip liner pencil, nailcare compositions, such as nail varnish, nail varnish remover, nail hardeners, or cuticle removers;
intimate care compositions, such as, for example, intimate washing lotions or intimate sprays;
footcare compositions, such as, for example, foot baths, foot powders, foot creams or foot balsams, especially deodorants and antiperspirants or compositions for removing hard skin;
sunscreens, such as sun milks, lotions, creams, oils, sunblocks or tropicals, pretanning preparations or aftersun preparations;
skin-tanning preparations, such as, for example, self-tanning creams;
depigmentation compositions, such as, for example, skin bleaching preparations or skin lightening compositions;
insect-repelling compositions (“repellants”), such as, for example, insect oils, lotions, sprays or sticks;
deodorants, such as deodorant sprays, pump sprays, deodorant gels, sticks or roll-ons;
antiperspirants, such as, for example, antiperspirant sticks, creams or roll-ons;
compositions for the cleansing and care of blemished skin such as, for example, syndets (solid or liquid), peeling or scrub preparations or peeling masks;
hair-removal compositions in chemical form (depilation), such as, for example, hair-removal powders, liquid depilatories, cream or paste depilatories, depilatories in gel form or aerosol foams;
shaving compositions, such as, for example, shaving soap, foaming shaving creams, nonfoaming shaving creams, foams and gels, preshave preparations for dry shaving, aftershaves or aftershave lotions;
fragrances, such as, for example, toilet waters (eau de Cologne, eau de toilette, eau de parfum, parfum de toilette, perfume), perfume oils or perfume creams;
compositions for dental care, denture care and oral care, such as, for example, tooth creams, gel tooth creams, tooth powders, mouthwash concentrates, antiplaque mouth-washes, prothesis cleaners or prothesis adhesives;
cosmetic compositions for hair treatment, such as, for example, hair cleansers in the form of shampoos, hair conditioners, haircare compositions, such as, for example, pre-treatment compositions, hair tonic, styling creams, styling gels, pomades, hair rinses, treatment packs, intensive hair treatments, compositions for shaping the hair, such as, for example, waving agents for producing permanent wave (hot-wave, mild-wave, cold-wave), hair-smoothing preparations, liquid hair-setting compositions, hair foams, hair sprays, blonding agents, such as, for example, hydrogen peroxide solutions, lightening shampoos, blonding creams, blonding powders, blonding pastes or oils, temporary, semipermanent or permanent hair colorants, preparations with self-oxidizing dyes, or natural hair colorants, such as henna or camomile.

An antimicrobial soap has, for example, the following composition:

| | | |
|---|---|---|
| 0.01 to 5% | by weight | of the compound of the formula (1) |
| 0.3 to 1% | by weight | of titanium dioxide, |
| 1 to 10% | by weight | of stearic stearic acid |

| | |
|---|---|
| ad 100% | of soap base, such as, for example, the sodium |
| | salts of tallow fatty acid and coconut fatty acid |
| | or glycerol. |

A shampoo has, for example, the following composition:

| 0.01 to 5% | by weight | of the compound of the formula (1), |
|---|---|---|
| 12.0% | by weight | of sodium laureth-2 sulphate, |
| 4.0% | by weight | of cocamidopropylbetaine, |
| 3.0% | by weight | of NaCl and |

| | |
|---|---|
| ad 100% | of water. |

A deodorant has, for example, the following composition:

| 0.01 to 5% | by weight | of the compound of the formula (1), |
|---|---|---|
| 60% | by weight | of ethanol, |
| 0.3% | by weight | of perfume oil, and |

| | |
|---|---|
| ad 100% | of water. |

The invention further provides an oral composition comprising 0.01 to 15% by weight, based on the total weight of the composition, of the compound of the formula (1) and orally acceptable auxiliaries.

Example of an oral composition:

| | | |
|---|---|---|
| 10% | by weight | of sorbitol, |
| 10% | by weight | of glycerol, |
| 15% | by weight | of ethanol, |
| 15% | by weight | of propylene glycol, |
| 0.5% | by weight | of sodium lauryl sulphate, |
| 0.25% | by weight | of sodium methyl cocyltaurate, |
| 0.25% | by weight | of polyoxypropylene/polyoxyethylene block |
| | | copolymer, |
| 0.10% | by weight | of peppermint flavouring, |
| 0.1 to 0.5% | by weight | of a compound of the formula (I), and |
| 48.6% | by weight | of water. |

The oral composition according to the invention can be, for example, in the form of a gel, a paste, a cream or an aqueous preparation (mouthwash).

In addition, the oral composition according to the invention can comprise compounds which release fluoride ions, which are effective against the formation of caries, e.g. inorganic fluoride salts, such as, for example, sodium fluoride, potassium fluoride, ammonium fluoride or calcium fluoride, or organic fluoride salts, such as, for example, amine fluorides, which are known under the trade name Olafluor.

In addition, the benzyl alcohol derivatives of the formula (1) used according to the invention are suitable for the treatment, in particular preservation, of textile fibre materials. The fibre materials are undyed and dyed or printed and are made of, for example, silk, wool, polyamide or polyurethanes, and in particular cellulosic fibre materials of all types. Such fibre materials are, for example, natural cellulose fibres, such as cotton, linen, jute and hemp, and also regenerated cellulose. Preferred suitable textile fibre materials are made of cotton.

The benzyl alcohols according to the invention are also suitable for the treatment, in particular for the antimicrobial finishing or preservation, of plastics, such as, for example, polyethylene, polypropylene, polyurethane, polyester, polyamide, polycarbonate, latex etc. Fields of use for these are, for example, floor coverings, plastic coatings, plastic container and packaging materials; kitchen and bathroom utensils (e.g. brushes, shower curtains; sponges, bathroom mats), latex, filter materials (air and water filters), plastic articles used in the medical sector, such as, for example, bandaging materials, syringes, catheters etc., so-called medical devices, gloves and mattresses.

Paper too, such as, for example, hygiene papers, can be antimicrobially finished with the benzyl alcohols according to the invention.

In addition, nonwovens, such as, for example, nappies, sanitary towels, panty liners, wipes for the hygiene and household sector, can be antimicrobially finished according to the invention.

In addition, the benzyl alcohols of the formula (1) are used in washing and cleaning formulations, such as, for example, in liquid and powder detergents or fabric softeners.

The benzyl alcohols can be used, in particular, also in household and all-purpose cleaners for the cleaning and disinfection of hard surfaces. A cleaner has, for example, the following composition:

| | |
|---|---|
| 0.01 to 5% | of the compound of the formula (1) |
| 3.0% | of octyl alcohol 4EO |
| 1.3% | of fatty alcohol C₈-C₁₀ polyglucoside |
| 3.0% | of isopropanol |
| ad 100% | of water. |

As well as the preservation of cosmetics and household products, the preservation and antimicrobial finishing of technical products and also use as biocide in technical processes is also possible, such as, for example, in the treatment of paper, in particular in paper-treatment liquors, printing thickeners made of starch or cellulose modifications, surface coatings and paints.

The benzyl alcohols of the formula (1) are also suitable for the antimicrobial treatment of wood and also for the antimicrobial treatment, preservation and finishing of leather.

In addition, the compounds according to the invention are suitable for protecting cosmetic products and household products against microbial decay.

The benzyl alcohols which can be used according to the invention are known compounds or novel compounds.

The novel compounds have the above formula 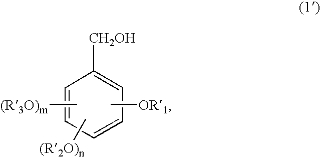
in which
R′₁,R′₂ and R′₃, independently of one another, are unsubstituted C₁-C₂₀alkyl, C₅₋₈cycloalkyl, C₃-C₂₀alkenyl or C₁-C₂₀alkyl, C₅₋₈cycloalkyl, C₃-C₂₀alkenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; C₂-C₂₀alkyl which is interrupted by one or more heteroatoms and/or may be substituted; C₂-C₂₀perfluoroalkyl; —(C₂-C₁₂alkylene)-Si-(tris-C₁₋₁₂alkyl); —(C₂-C₁₂alkylene)-Si-(di-C₁₋₁₂alkyl)-C₃-C₁₂alkenyl; or —(CH₂)ₓ(CHCH₃)_{y}(C(CH₃)₂)_{z}-A;
A is unsubstituted phenyl, naphthyl or C₄-C₈cycloalkyl or phenyl, naphthyl or C₄-C₈cycloalkyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; or —CH₂CH₂(CH₂CH₂O)ₒ—B;
B is unsubstituted C₁-C₄alkyl or phenyl or C₁-C₄alkyl or phenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂;
o is an integer from 0-10;
n and m, independently of one another, are 0 or 1; and
x, y and z, independently of one another are 0 to 12;
with the proviso that
if n and m are O, R′₁ is not an alkyl group,
if R′₁ is methyl or ethyl and either n or m is 1, then R′₂ or R′₃ is not a methyl or benzyl group, and
if R′₁ is a linear C₁₂alkyl group and either n or m is 1, then R′₂ or R′₃ is not a linear C₁₂alkyl group.

The examples below, which do not limit the invention, are used for illustration.

### EXAMPLE 1

In a reaction vessel with attached condenser and stirrer, 10 g (82 mmol) of 4-hydroxybenzyldehyde and 11.3 g (82 mmol) of dried potassium carbonate are initially introduced into 60 ml of dry dimethylformamide, and 17.4 g (90 mmol) of n-octyl bromide are added. The mixture is stirred for 18 hours at 80° C. After the solvent has been stripped off at 10⁻² mbar, the residue is taken up in 100 ml of water and 50 ml of petroleum ether, the organic phase is separated off, dried and then the solvent is stripped off. The intermediate can be used subsequently without further purification steps.

For the reduction, 2.5 g (67 mmol) of lithium aluminium hydride are initially introduced into 200 ml of diethyl ether, and, at room temperature, the solution of 15.7 g (67 mmol) of the precursor in 50 ml of diethyl ether is added dropwise thereto. The reaction mass is refluxed for 6 hours and, after cooling, carefully transferred dropwise to 200 ml of water. Following acidification of the aqueous phase with concentrated hydrochloric acid to pH 2, the organic phase is separated off, washed with water until neutral and evaporated. The crystalline residue is recrystallized from a small amount of n-hexane.

The resulting colourless crystals which correspond to the compound of the formula 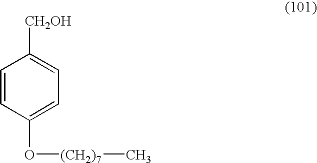
(=compound (9) in Table 1), are obtained in a yield of 62%.

The purity is 97 area % LC(254 nm).

The recorded ¹H/¹³C-NMR spectra confirm the structure.

The other compounds in Table 1 can be prepared analogously to Synthesis Example 1 using the respective starting materials.

Determination of the Minimum Inhibition Concentration (MIC Value) in Microtitre Plates

Nutrient Medium:

Casein-soy flour-peptone broth for the preparation of the precultures of the test bacteria and yeast.

Examples of Test Microbes:

Bacteria: Staphylococcus aureus ATCC 6538
Escherichia coli NCTC 8196 (=EC)
   Procedure:

The test substances are predissolved in dimethyl sulphoxide (DMSO) and tested in a dilution series of 1:2.

The bacteria are cultivated overnight in CASO broth and sponged off with 10 ml of 0.85% sodium chloride solution (+0.1% TritonX-100).

All of the test microbes are adjusted to a microbial count of 1-5×10⁶ CFU/ml with 0.85% sodium chloride solution.

The test substances are prepipetted at 8 μl per well into microtitre plates.

Prediluted microbial suspensions are diluted 1:100 in CASO broth and added at 192 μl per well to the test substances.

The test mixtures are incubated at 37° C. for 48 hours.

After incubation, the growth is determined by reference to the clouding of the test mixtures (optical density) at 620 nm in a microplate reader.

The minimum inhibition concentration (MIC value) is the concentration of substance at which (compared with the growth control) a significant growth inhibition (<20% growth) of the test microbes is established.

One microtitre plate is prepared per test microbe and substance concentration. All of the substances are tested in duplicate.

Table 2 lists the results. The compounds correspond to those in Table 1:

TABLE 2

| | MIC [ppm] | |
|---|---|---|
| | Staphylococcus | MIC [ppk] |
| Example | aureus | Escherichia coli |
| (3) | 60 | >120 |
| (4) | 120 | >120 |
| (5) | 60 | >120 |
| (6) | 120 | >120 |
| (7) | 120 | >120 |
| (8) | 60 | 60 |
| (9) | 30 | 60 |
| (10) | 120 | 60 |
| (11) | 15 | 15 |
| (12) | 60 | 60 |
| (13) | 60 | >120 |
| (14) | 15 | 30 |
| (15) | 120 | 120 |
| (16) | 15 | >120 |
| (17) | 60 | 60 |
| (18) | 120 | 60 |
| (19) | 120 | 60 |
| (20) | 60 | 60 |
| (21) | 30 | 30 |
| (22) | 60 | 60 |
| (23) | 60 | 60 |
| (24) | 60 | 60 |
| (25) | 120 | 120 |
| (26) | 15 | 15 |
| (27) | 30 | 60 |
| (28) | 30 | 30 |
| (29) | 120 | >120 |
| (30) | 60 | 60 |
| (31) | 120 | >120 |
| (32) | 60 | >120 |
| (33) | 15 | >120 |
| (34) | 120 | 60 |
| (35) | 60 | 120 |

1. A method for the antimicrobial treatment of a surface, which comprises contacting said surface with a compound of the formula 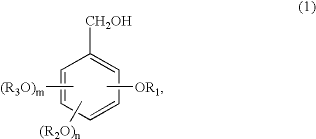
in which
R₁,R₂ and R₃, independently of one another, are unsubstituted C₁-C₂₀alkyl, C₅-₈cycloalkyl, C₃-C₂₀alkenyl or C₁-C₂₀alkyl, C₅-₈cycloalkyl, C₃-C₂₀alkenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; C₂-C₂₀alkyl which is interrupted by one or more heteroatoms and/or may be substituted; C₂-C₂₀perfluoroalkyl; —(C₂-C₁₂alkylene)-Si-(tris-C₁₋₁₂alkyl);
(C₂-C₁₂alkylene)-Si-(di-C₁₋₂alkyl)-C₃-C₁₂alkenyl; or —(CH₂)ₓ(CHCH₃)_{y}(C(CH₃)₂)_{z}-A;
A is unsubstituted phenyl, naphthyl or C₄-C₈cycloalkyl or phenyl, naphthyl or C₄-C₈cycloalkyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; or —CH₂CH₂(CH₂CH₂O)ₒ—B;
B is unsubstituted C₁-C₄alkyl or phenyl or C₁-C₄alkyl or phenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂;
o is an integer from 0-10;
n and m, independently of one another, are 0 or 1; and
x, y and z, independently of one another, are 0 to 12;
and mixtures thereof and salts thereof,
where compounds of the formula (1) in which
R₁ and R₂ or R₁ and R₃ or R₂ and R₃ are methyl or ethyl are not included.

2. A method according to claim 1, wherein
R₁, R₂ and R₃, independently of one another, are unsubstituted C₅-C₁₆alkyl, C₃-C₆cycloalkyl or C₃-C₁₆alkenyl or C₅-C₁₆alkyl, C₃-C₆cycloalkyl or C₃-C₁₆alkenyl substituted by C₁-C₄alkyl, C₁-C₄alkoxy, hydroxy, phenyl, halogen, CN, COOH, COO—C₁-C₂-alkyl, amino, NHC₁-C₂₀alkyl, N(C₁-C₂₀alkyl)₂; C₃-C₁₆alkyl which is interrupted by one or more heteroatoms from the group O, N or S; C₁-C₁₆perfluoroalkyl; —(C₁-C₁₂alkylene)-Si-(tris-C₁₋₁₂alkyl); —(C₁-C₆alkylene)-Si-(di-C₁-C₂alkyl)allyl; —(CH₂)ₓ(CHCH₃)_{y}(C(CH₃)₂)_{z}-A; or —CH₂CH₂(CH₂CH₂O)ₒ—B;
A is unsubstituted phenyl or C₅-C₆cycloalkyl or phenyl or C₅-C₆cycloalkyl substituted by C₁-C₄alkyl, C₁-C₄alkoxy, hydroxy, phenyl, halogen, —CN, —COOH, —COO—C₁-C₂alkyl, amino, NHC₁-C₂₀alkyl or N(C₁-C₂galkyl)₂;
B is unsubstituted C₁-C₂alkyl or phenyl or C₁-C₂alkyl or phenyl substituted by C₁-C₄alkyl, C₁-C₄alkoxy, hydroxy, phenyl, halogen, —CN, —COOH, —COO—C₁-C₂alkyl, amino, NHC₁-C₂₀alkyl or N(C₁-C₂₀alkyl)₂;
x, y and z, independently of one another, are an integer from 0 to 4;
o is an integer from 0 to 5; and
n and m, independently of one another, are 0 or 1.

3. A method according to claim 1, wherein compounds of the formula 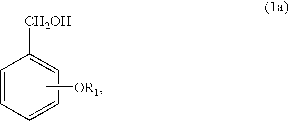
in which
R₁ is —(CH₂)₁₋₃Si(CH₃)₃; —CH₂Si(CH₃)₂(CH₂CH═CH₂); —CH₂SiC₆H₅; —(CH₂)₅CH₃; —(CH₂)₇CH₃; —CH(CH₂CH₃)₂; or —CH(CH₃)(CH₂)₄CH₃; are used.

4. A method according to claim 1, wherein compounds of the formulae 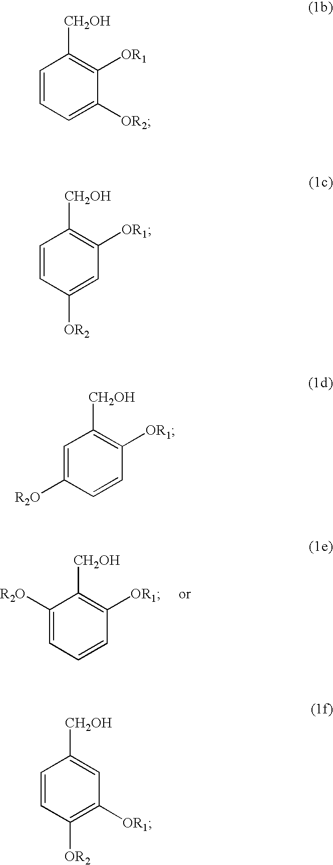
in which
R₁ is —(CH₂)₁₋₃Si(CH₃)₃; —CH₂Si(CH₃)₂(CH₂CH═CH₂); —CH₂Si(CH₃)₂C₆H₅; linear C₄-C₁₀alkyl; —CH (CH₂CH₃)₂; —CH(CH₃)(CH₂)₄CH₃; —CH₂CH₂OC₆H₅; cyclohexyl; or —CH₂C₆H₅;
R₂ is —C₁-C₄alkyl; —(CH₂)₁₋₃Si(CH₃)₃; —CH₂Si(CH₃)₂(CH₂CH═CH₂); —CH₂Si(CH₃)₂C₆H₅; —(CH₂)₃CH₃; —(CH₂)₅CH₃; —(CH₂)₇CH₃; —CH(CH₂CH₃)₂; —CH(CH₃)(CH₂)₄CH₃; —CH₂CH₂OC₆H₅; cyclohexyl; or —CH₂C₆H₅; are used.

5. A method according to claim 4, wherein
R₁ is —CH₂Si(CH₃)₃—(CH₂)₃Si(CH₃)₃; —CH₂Si(CH₃)₂(CH₂CH═CH₂); —CH₂Si(CH₃)₂C₆H₅; —(CH₂)₅CH₃; —(CH₂)₇CH₃; —CH(CH₂CH₃)₂; —CH(CH₃)(CH₂)₄CH₃; —CH₂CH₂OC₆H₅; —(CH₂)₃CH₃; cyclohexyl; or —CH₂C₆H₅; and
R₂ is methyl.

6. A process for the preparation of the compounds of the formula (1) according to claim 1, which comprises reacting a compound of the formula (2a) 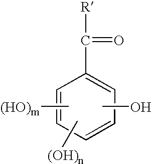
with a halide Hal-R₁ or a mixture of Hal-R₁/Hal-R₂, Hal-R₁/Hal-R₃, Hal-R₂/Hal-R₃ or Hal-R₁/Hal-R₂/Hal-R₃ to give the compound of the formula (2b) 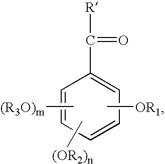
and then converting it, by reduction, into the compound of the formula (1) according to claim 1 in accordance with the following scheme: 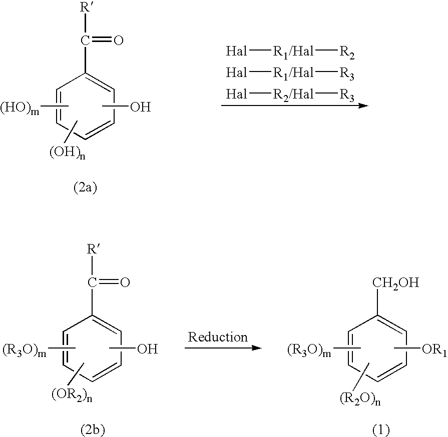
in which
R′ is hydrogen or O—C₁-C₅alkyl, and
R₁, R₂, R₃, m and n have the meanings given in claim 1.

7. A method according to claim 1, wherein the compound of the formula (1) is used for the antimicrobial treatment, deodorization and disinfection of the skin, mucosae and hair.

8. A method according to claim 7, wherein the compound of the formula (1) is used for disinfection and deodorization.

9. A method according to claim 1, wherein of the compound of the formula (1) is used for the treatment of textile fibre materials.

10. A method according to claim 9, wherein the compound of the formula (1) is used for preservation.

11. A method according to claim 1, wherein the compound of the formula (1) is used in washing and cleaning formulations.

12. A method according to claim 1, wherein the compound of the formula (1) is used for the antimicrobial finishing and preservation of plastics, paper, nonwovens, wood or leather.

13. A method according to claim 1, wherein the compound of the formula (1) is used for the antimicrobial finishing and preservation of technical products selected from the group consisting of, printing thickeners made of starch or cellulose modifications, surface coatings and paints.

14. A method according to claim 1, wherein the compound of the formula (1) is used as a biocide in technical processes.

15. A bodycare composition comprising 0.01 to 15% by weight, based on the total weight of the composition, of the compound of the formula (1) according to claim 1 and cosmetically acceptable auxiliaries.

16. An oral composition comprising 0.01 to 15% by weight, based on the total weight of the composition, of the compound of the formula (1) according to claim 1 and orally acceptable auxiliaries.

17. A compound of the formula 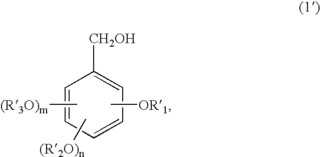
in which
R′₁,R′₂ and R′₃, independently of one another, are unsubstituted C₁-C₂₀alkyl, C₅₋₈cycloalkyl, C₃-C₂₀alkenyl or C₁-C₂₀alkyl, C₅₋₈cycloalkyl, C₃-C₂₀alkenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; C₂-C₂₀alkyl which is interrupted by one or more heteroatoms and/or may be substituted; C₂-C₂₀perfluoroalkyl; —(C₂-C₁₂alkylene)-Si-(tris-C₁₋₁₂alkyl); —(C₂-C₁₂alkylene)-Si-(di-C₁₋₁₂alkyl)-C₃-C₁₂alkenyl; or —(CH₂)ₓ(CHCH₃)_{y}(C(CH₃)₂)_{z}-A;
A is unsubstituted phenyl, naphthyl or C₄-C₈cycloalkyl or phenyl, naphthyl or C₄-C₈cycloalkyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂; or —CH₂CH₂(CH₂CH₂O)ₒ—B;
B is unsubstituted C₁-C₄alkyl or phenyl or C₁-C₄alkyl or phenyl substituted by C₁-C₄alkyl, C₁-C₄alkoxy, hydroxy, phenyl, halogen, —CN, —COOH, —COO—C₁-C₂alkyl, amino, —NHC₁-C₂₀alkyl or —N(C₁-C₂₀alkyl)₂;
o is an integer from 0-10;
n and m, independently of one another, are 0 or 1; and
x, y and z, independently of one another are 0 to 12;
and mixtures thereof and salts thereof,
with the proviso that
if n and m are 0, R′₁ is not an alkyl group,
if R′₁ is methyl or ethyl and either n or m is 1, then R′₂ or R′₃ is not a methyl or benzyl group, and
if R′₁ is a linear C₁₂alkyl group and either n or m is 1, then R′₂ or R′₃ is not a linear C₁₂alkyl group.
